(19) United States
(12) Patent Application Publication
(10) Pub. No.: US 2023/0398586 A1
(43) Pub. Date: Dec. 14, 2023

(54) METHOD FOR DECOMPOSING PLANT MATTER

(71) Applicant: DANSTAR FERMENT AG, Zug (CH)

(72) Inventors: Jean-Marc SANCHEZ, Daux (FR); Matthieu MOREL, Villeneuve les Avignon (FR); Selma ROGALSKA, Konstancin- Jeziorna (PL)

(73) Assignee: DANSTAR FERMENT AG, Zug (CH)

(21) Appl. No.: 18/249,919

(22) PCT Filed: Oct. 20, 2021
(86) PCT No.: PCT/EP2021/079129

§ 371 Date: Apr. 20, 2023

(30) Foreign Application Priority Data
Oct. 21, 2020 (EP) 20306255.9

(51) Int. Cl. B09B 3/60; C12N 1/06

(52) U.S. Cl. CPC B09B 3/60; C12N 1/063; B09B 2101/85

(57) ABSTRACT

The present disclosure provides a method for accelerating decomposition of an organic or plant matter comprising contacting the organic or the plant matter with an effective amount of soluble yeast cell wall derivatives as an active ingredient for degrading the organic or plant matter to produce a decomposition product.

### TECHNOLOGICAL FIELD

The present disclosure relates to a method of accelerating the decomposition of organic or plant matter.

### BACKGROUND

Once crops such as cereals, corn, soybeans, cotton or sugar cane are harvested, remnants of the crops known as stubble, remain in the field. Similarly, in orchards or vineyards, leaves fall from the trees to the ground in the fall.

Spores that overwinter in organic/bioorganic or plant matter or leaf litter on the ground surface cause primary infection in spring. If the overwintering inoculum (i.e. the initial inoculum) is reduced or eliminated, the potential incidence of the disease, such as apple scab, is reduced.

Natural decomposition of plants, stubble or leaves on the surface of the ground usually does not eliminate spore development sufficiently during winter months because of temperature, moisture and nutrient constraints.

The ground can be kept free of leaves by various methods before new bud break in the spring. Fallen leaves can be raked, blown or vacuumed from beneath the trees and then removed before the spores have a chance to mature and develop.

Another strategy for managing primary inoculum is through the destruction of plant and/or leaf litter in the fall or spring via shredding, litter degrading compounds or biological control agents. This idea is to hasten the decomposition of the plant (e.g. plant matter) or leaf litter and, hence, the decomposition of overwintering inoculum in an effort to reduce the potential spores to a level that allows one to delay, reduce or eliminate the need for fungicide to manage disease such as apple scab. In other words, the destruction of plant matter or leaf litter reduces the inoculum pressure and effectively delays or eliminates the exponential increase in disease.

It is known in the art that urea has been highly effective in reducing pseudothecia and ascospore production of V. inaequalis leaves affected by apple scab. The effect of urea has been attributed to acceleration of decomposition of leaves by microorganisms. Furthermore, Burchill (1965) found that application of urea softened leaf tissue and made them more palatable to earthworms. More particularly, a foliar application of a 5% urea solution in autumn just before leaf drop will speed natural decomposition of leaves and help to deactivate, for example, the scab fungus. Another equally effective approach is to apply urea to leaves directly on the ground surface after they have dropped in the fall. The effectiveness of urea may vary from year to year in function, for example, of the weather conditions.

However, because urea is a synthetic compound, organic growers are not permitted to use it in their management programs. This limits sanitation (e.g. apple scab sanitation) options exclusively to removal or shredding the plant or leaf litter, practices which are often time consuming and difficult to implement. As a consequence, organic orchards often have poor primary inoculum management, resulting in epidemics. This is also true for conventional farming where the addition of nitrogen is undesirable for horticultural reasons.

In a need to find alternative effective sanitation methods in apple orchards, studies were conducted using yeast extract preparations to supress pseudothecial development and/or limit the discharge of ascospores of V. inaequalis. Results demonstrated that the application of 30% and 60% yeast extract to leaf litter depots showed the greatest efficacy and significantly reduced ascospore discharge by 99%. The efficacy of the treatment did not differ from treatment with urea 5%. Furthermore, leaf decay was accelerated in the plots treated with yeast extract compared with untreated control plots (Porsche et al., 2016; Porsche et al., 2017).

The above highlights limitations in primary inoculum management in both organic and conventionally managed orchards or fields. Therefore, there is a need for researches that explore new compounds or compositions that can be used by growers for potentially substituting urea applications which new compounds or compositions can improve decomposition of the leaves and further reduce fungi primary inoculum. Indeed, it was found, for example, that inoculum reduction of Venturia inaequalis was owing to increases in leaf litter decomposition.

### BRIEF SUMMARY

The present disclosure is directed to a new method for decomposing or for accelerating the decomposition or degradation of organic or plant matter and thereby reducing the overwintering potential of the primary inoculum.

The present disclosure relates to a method for accelerating decomposition of organic or plant matter, comprising contacting the organic or the plant matter with an effective amount of soluble hydrolysed yeast cell wall derivatives as an active ingredient for degrading the organic or plant matter to produce a decomposition product. The soluble hydrolysed yeast cell wall derivatives may be soluble enzymatically-treated yeast cell wall derivatives. The soluble enzymatically-treated yeast cell wall derivatives may be soluble protease-treated yeast cell wall derivatives. The soluble hydrolysed yeast cell wall derivatives may comprise or consist of a soluble mannan-oligosaccharide fraction. The yeast-derived soluble mannan-oligosaccharide fraction may comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide, preferably at least about 20% or at least about 30% mannan-oligosaccharide; and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins. The organic or plant matter may comprise monocotyledonous plant matter or dicotyledonous plant matter, preferably leaves, tree foliage, leaf litter and/or crop residues. For example, the organic or plant matter may comprise leaves, leaf litter and/or crop residues of cereals crops, sugar cane, corn, vines, vegetable crops or fruit trees, such as leaves and/or leaf litter from apple trees. The soluble hydrolysed yeast cell wall derivatives may be contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably at about between 30% and 75% leaf fall. The soluble hydrolysed yeast cell wall derivatives may be contacted with plant matter on the ground at about 30%, 35%, 40%, 45%, 50%, 55%, 60%, 65%, 70%, 75%, 80%, 85%, 90% or 95% leaf fall. The soluble hydrolysed yeast cell wall derivatives may be used alone or in combination with urea.

In a second aspect, the disclosure relates to a use of soluble hydrolysed yeast cell wall derivatives for degrading organic or plant matter to produce a decomposition product. The soluble hydrolysed yeast cell wall derivatives may be soluble enzymatically-treated yeast cell wall derivatives. The soluble enzymatically-treated yeast cell wall derivatives may be soluble protease-treated yeast cell wall derivatives. The soluble hydrolysed yeast cell wall derivatives may comprise or consist of a soluble mannan-oligosaccharide fraction. The yeast-derived soluble mannan-oligosaccharide fraction may comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide, preferably at least about 20% or at least about 30% mannan-oligosaccharide;

and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins. The organic or plant matter may comprise monocotyledonous plant matter or dicotyledonous plant matter, preferably leaves, tree foliage, leaf litter and/or crop residues. The organic or plant matter may comprise leaves, leaf litter and/or crop residues of cereals crops, sugar cane, corn, vines, vegetable crops or fruit trees, such as leaves and/or leaf litter from apple trees. The soluble hydrolysed yeast cell wall derivatives may be contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably at about between 30% and 75% leaf fall. The soluble hydrolysed yeast cell wall derivatives may be contacted with plant matter on the ground at about 30%, 35%, 40%, 45%, 50%, 55%, 60%, 65%, 70%, 75%, 80%, 85%, 90% or 95% leaf fall.

In any of the aspects of the disclosure discussed herein, the soluble hydrolysed yeast cell derivatives may be obtainable by hydrolysing a yeast cell wall fraction. The soluble hydrolysed yeast cell derivatives may be obtainable by hydrolysing a yeast cell wall fraction with an enzyme, optionally with a protease. The soluble hydrolysed yeast cell derivatives may be obtainable by (i) subjecting a yeast cell wall fraction to an enzymatic treatment to obtain insoluble yeast cell wall derivatives comprising a β-glucan enriched cell wall fraction and a yeast-derived soluble mannan-oligosaccharide fraction, and (ii) separating said β-glucan enriched cell wall fraction from said yeast-derived soluble mannan-oligosaccharide fraction.

The enzymatic treatment may be protease treatment. The soluble hydrolysed yeast cell wall derivatives may be obtainable by a method comprising the following steps (i) providing a yeast cell material from a species from the genera Saccharomyces, Kluyveromyces, Hanseniaspora, Metschnikowia, Pichia, Starmerella, Torulaspora or Candida, preferably wherein the yeast is S. cerevisiae, (ii) subjecting said yeast material to autolysis and/or enzyme assisted hydrolysis for a sufficient time to obtain a yeast autolysate and/or a yeast hydrolysate comprising a soluble yeast extract fraction and an insoluble yeast cell wall fraction, (iii) subjecting said yeast autolysate or said yeast hydrolysate to separation to separate the soluble yeast extract fraction from the insoluble yeast cell wall fraction, (iv) recovering the yeast cell wall fraction and discarding the soluble yeast extract fraction, (v) subjecting the yeast cell wall fraction to an enzymatic treatment with a protease to obtain yeast cell wall derivatives comprising a β-glucan enriched cell wall fraction and a yeast-derived soluble mannan-oligosaccharide fraction, (vi) separating said β-glucan enriched cell wall fraction from said yeast-derived soluble mannan-oligosaccharide fraction, and (vii) recovering said yeast-derived soluble mannan-oligosaccharide fraction.

In another aspect, the disclosure relates to a method for reducing the inoculum of an overwintering pathogenic fungus, comprising contacting organic matter or plant matter with an effective amount of soluble hydrolysed yeast cell wall derivatives as an active ingredient for degrading the organic or plant matter to produce a decomposition product and to reduce the inoculum of the overwintering pathogenic fungus. The organic or plant matter may comprise monocotyledonous plant matter or dicotyledonous plant matter, preferably leaves, tree foliage, leaf litter and/or crop residues. The organic or plant matter may comprise leaves, leaf litter and/or crop residues of cereals crops, sugar cane, corn, vines, vegetable crops or fruit trees, such as leaves and/or leaf litter from apple trees or grape vines. The overwintering pathogenic fungus may be apple scab (e.g. Ventirua inaequalis) or powdery mildew (e.g. Erysiphe necator). The soluble hydrolysed yeast cell wall derivatives may be contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably at about between 30% and 75% leaf fall. The soluble hydrolysed yeast cell wall derivatives may be contacted with plant matter on the ground at about 30%, 35%, 40%, 45%, 50%, 55%, 60%, 65%, 70%, 75%, 80%, 85%, 90% or 95% leaf fall. The soluble hydrolysed yeast cell wall derivatives may be soluble enzymatically-treated yeast cell wall derivatives. The soluble enzymatically-treated yeast cell wall derivatives may be soluble protease-treated yeast cell wall derivatives. The soluble hydrolysed yeast cell wall derivatives may comprise or consist of a soluble mannan-oligosaccharide fraction. The yeast-derived soluble mannan-oligosaccharide fraction may comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide, preferably at least about 20% or at least about 30% mannan-oligosaccharide; and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins.

In a further aspect, the disclosure relates to the use of soluble hydrolysed yeast cell wall derivatives for reducing the inoculum of an overwintering pathogenic fungus in organic or plant matter. The organic or plant matter may comprise monocotyledonous plant matter or dicotyledonous plant matter, preferably leaves, tree foliage, leaf litter and/or crop residues.

The organic or plant matter may comprise leaves, leaf litter and/or crop residues of cereals crops, sugar cane, corn, vines, vegetable crops or fruit trees, such as leaves and/or leaf litter from apple trees or grape vines. The overwintering pathogenic fungus may be apple scab (e.g. Ventirua inaequalis) or powdery mildew (e.g. Erysiphe necator). Where the organic or plant matter comprises leaves and/or leaf litter, the soluble hydrolysed yeast cell wall derivatives (i) may be used on tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably at about between 30% and 75% leaf fall; and/or (ii) may be used on the ground at about 30%, 35%, 40%, 45%, 50%, 55%, 60%, 65%, 70%, 75%, 80%, 85%, 90% or 95% leaf fall. The soluble hydrolysed yeast cell wall derivatives may be soluble enzymatically-treated yeast cell wall derivatives. The soluble enzymatically-treated yeast cell wall derivatives may be soluble protease-treated yeast cell wall derivatives. The soluble hydrolysed yeast cell wall derivatives may comprise or consist of a soluble mannan-oligosaccharide fraction. The yeast-derived soluble mannan-oligosaccharide fraction may comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide, preferably at least about 20% or at least about 30% mannan-oligosaccharide; and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins.

In a first aspect, the present disclosure concerns a method for accelerating decomposition of organic or plant matter comprising contacting the organic or the plant matter with an effective amount of soluble yeast cell wall derivatives as an active ingredient for degrading the organic or plant matter to produce a decomposition product. In an embodiment, the soluble yeast cell wall derivatives comprise a yeast-derived soluble mannan-oligosaccharide fraction. In particular, the yeast-derived soluble mannan-oligosaccharide fraction comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan, preferably wherein the yeast-derived soluble mannan-oligosaccharide product comprises at least about 20% or at least about 30% mannan; and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably wherein the yeast-derived soluble mannan-oligosaccharide product comprises at least about 30% or 35% of proteins. In still another embodiment, the organic or plant matter comprises monocotyledonous plant matter or dicotyledonous plant matter. In another embodiment, the monocotyledonous plant matter or dicotyledonous plant matter comprises leaves, tree foliage, leaf litter and/or crop residues of, for example, cereals crops, sugar cane, corn, vines, vegetable crops or fruit trees. In an embodiment, the organic or plant matter comprises leaves and/or leaf litter from apple trees. In an embodiment, the soluble yeast cell wall derivatives of the present disclosure are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble yeast cell wall derivatives are contacted with tree foliage or leaves at about between 30% and 75% leaf fall; and/or the soluble yeast cell wall derivatives are contacted with plant matter on the ground at about 30%, 35%, 40%, 45%, 50%, 55%, 60%, 65%, 70%, 75%, 80%, 85%, 90% or 95% leaf fall. In another embodiment, the soluble yeast cell wall derivatives are used in alone or in combination with urea.

In a second aspect, the present disclosure concerns the use of soluble yeast cell wall derivatives for degrading organic or plant matter to produce a decomposition product. In an embodiment, the soluble yeast cell wall derivatives are a yeast-derived soluble mannan-oligosaccharide product which comprises (a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan, preferably at least about 20% or at least about 30% mannan; and (b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins. In an embodiment, the organic or plant matter comprises monocotyledonous plant matter or dicotyledonous plant matter. In another embodiment, the monocotyledonous plant matter or dicotyledonous plant matter comprises leaves, tree foliage, leaf litter and/or crop residues of, for example, cereals crops, sugar cane, corn, vines, vegetable crops or fruit trees. In an embodiment, the organic or plant matter comprises leaves and/or leaf litter from apple trees. In an embodiment, the soluble yeast cell wall derivatives are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble yeast cell wall derivatives are contacted with tree foliage or leaves at about between 30% and 75% leaf fall; and/or (b) the soluble yeast cell wall derivatives are contacted with plant matter on the ground at about 30%, 35%, 40%, 45%, 50%, 55%, 60%, 65%, 70%, 75%, 80%, 85%, 90% or 95% leaf fall. In a specific embodiment, the soluble yeast cell wall derivatives of the present disclosure are obtained by a method comprising the following steps: i. providing a yeast cell material from a species from the genera Saccharomyces, Kluyveromyces, Hanseniaspora, Metschnikowia, Pichia, Starmerella, Torulaspora or Candida, preferably wherein the yeast is S. cerevisiae; ii. subjecting said yeast material to autolysis and/or enzyme assisted hydrolysis for a sufficient time to obtain a yeast autolysate and/or a yeast hydrolysate comprising a soluble yeast extract fraction and an insoluble yeast cell wall fraction; iii. subjecting said yeast autolysate or said yeast hydrolysate to separation to separate the soluble yeast extract fraction from the insoluble yeast cell wall fraction; iv. recovering the yeast cell wall fraction and discarding the soluble yeast extract fraction; v. subjecting the yeast cell wall fraction to an enzymatic treatment with a protease to obtain yeast cell wall derivatives comprising a β-glucan enriched cell wall fraction and a yeast-derived soluble mannan-oligosaccharide fraction; vi. separating said β-glucan enriched cell wall fraction from said yeast-derived soluble mannan-oligosaccharide fraction; and vii. recovering said yeast-derived soluble mannan-oligosaccharide fraction.

### FIGURE

Having thus generally described the nature of the invention, reference will now be made to the accompanying drawing, showing by way of illustration, a preferred embodiment thereof, and in which:

FIG. 1 is a flowchart of a general process to produce the soluble yeast cell wall derivatives in accordance with the present disclosure.

### DETAILED DESCRIPTION

The present disclosure concerns the use of soluble yeast cell wall derivatives as an active ingredient for decomposing organic or plant matter. The management of organic or plant matter, as for example leaf litter, is correlated with a reduction of the primary and consequently the secondary inoculum which will postpone the initial infection of the disease (e.g. the initial infection of Venturia inaequalis). The use of soluble yeast cell wall derivatives can reduce disease pressure and decrease the need for frequent applications of high rates fungicides by decomposing the plant or leaf litter on the ground surface.

As used herein, the soluble yeast cell wall derivatives of the present disclosure used as an active ingredient comprises yeast-derived soluble mannan-oligosaccharides linked to peptide complex or a mannan-oligosaccharide (MOS) extract. The soluble yeast cell wall derivatives of the present disclosure come from the soluble fraction obtained from hydrolysed yeast cell walls. The soluble yeast cell wall derivatives of the present disclosure are from a distinct nature and source than those present in the yeast extract (i.e. yeast soluble fraction).

The soluble yeast cell wall derivatives of the present disclosure are soluble derivatives of the yeast cell wall. The soluble yeast cell wall derivative may be a soluble hydrolysed yeast cell wall derivative such as a soluble fraction of hydrolysed yeast cell walls, i.e. a soluble fraction obtained by hydrolysing yeast cell walls. The soluble yeast cell wall derivative may be a soluble protease-treated yeast cell wall derivative such as a soluble fraction of protease-treated yeast cell walls, i.e. a soluble fraction obtained by treating yeast cell walls with a protease. The soluble yeast cell wall derivative may be a soluble extract of hydrolysed yeast cell wall, e.g. protease-treated yeast cell wall.

As used herein, the term “yeast extract” refers to the content or the intracellular components of the yeast cells, with the yeast cell wall removed, said content being obtained by any suitable extraction process known to those skilled in the art. For example, the yeast extract can be obtained by autolysis or plasmolysis. The yeast extract refers to the soluble fraction.

As used herein, the “yeast cell walls” are obtained by separation of the envelope and the rest of the yeast cell. In other words, the “yeast cell wall” fraction or the insoluble fraction corresponds to the envelopes of the yeast cells excluding the contents of the cells, i.e the intracellular components of the yeast cells. The yeast cell walls consist predominantly of beta-glucans and mannans.

Mannan is a polymer composed of mannose units. In yeasts, mannan is associated with protein in both the external surface of the yeast cell wall, as a muscilaginous polysaccharide, and in the inner cell membrane. It generally accounts for about 20-50% of the dry weight of the cell wall. Mannan is linked to a core-peptide chain as an oligomer or polymer. The complex contains about 5-50% proteins. Oligomeric mannan is bonded directly to serine and threonine residue of the peptide, whereas polymeric mannan is bonded to aspargine via N-acetylglucosamine. In the manno-protein complex, the mannose units are linked by α-1,6, α-1,2 and α-1,3-linkages. Mannan-oligosaccharides linked to smaller peptides complexes can be released from yeast cell walls by proteolytic action.

Such mannan preparations can be prepared from microorganisms, such as yeast, using different hydrolysis steps to release mannans from the yeast cell walls.

Suitable yeast species as a source of mannans include, but are not limited to, yeast strains of Saccharomyces, Kluyveromyces, Hanseniaspora, Metschnikowia, Pichia, Starmerella, Torulaspora or Candida. In an embodiment, yeast strains are from Saccharomyces cerevisiae (including baker's yeast strains and brewer's yeast strains), Kluyveromyces fragilis, or Candida strains, such as Candida utilis, and combinations thereof. In an embodiment, the yeast used in the context of the present disclosure is S. cerevisiae. These yeast strains can be produced either by batch fermentation or continuous fermentation. In an embodiment, a yeast cream is used in the process to produce the yeast-derived soluble mannan-oligosaccharides.

Specifically, the process to produce the yeast-derived soluble mannan-oligosaccharides in accordance to the present disclosure starts with the generation of yeast cell wall preparations produced from microorganisms including, but not limited to, yeast.

In an exemplified embodiment, the process includes a first step of autolysis or hydrolysis of yeast or cream yeast. The autolysis or hydrolysis may suitably be carried out at a pH of at least 4, particularly at least 4.5, and more particularly at least 5. The autolysis or hydrolysis may suitably be carried out at a pH of less than 8, particularly less than 7, and even more particularly less than 6. The temperature for carrying out the autolysis or hydrolysis may suitably be at least 30° C., at least 35° C., at least 40° C., at least 45° C., at least 50° C., at least C, at least 60° C. or at least 65° C. The temperature for carrying out the autolysis or hydrolysis may suitably be less than 65° C. In an embodiment, the temperature for carrying out the autolysis is between at least 45° C. and 65° C. The autolysis or hydrolysis may suitably be carried out for at least 2, hours, 3 hours, 4 hours, 5 hours, 6 hours, 7 hours, 8 hours, 9 hours, 10 hours, particularly at least 14 hours, and more particularly at least 24 hours. The autolysis or hydrolysis may suitably be carried out for less than 60 hours, particularly less than 48 hours, and even more particularly less than 36 hours. In an embodiment, the autolysis or hydrolysis is carried out for at least 12 to 30 hours. To increase the efficiency of the autolysis or hydrolysis process, exogenous enzymes such as proteolytic enzymes can be used. The yeast autolysate or yeast hydrolysate is then separated, suitably by centrifugation, to produce a yeast extract fraction (i.e. the soluble fraction) and a yeast cell wall fraction (i.e. the insoluble fraction). The yeast cell wall fraction is recovered while the yeast extract fraction is discarded. The recovered cell wall fraction is further treated with a proteolytic enzyme at a pH of at least 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 or 10. The recovered cell wall fraction is further treated with a proteolytic enzyme at a pH of at least 8.0, 8.1, 8.2, 8.3, 8.4 or 8.5. The proteolytic treatment may suitably be carried out at a temperature of at least 40° C., at least 45° C., at least 50° C., at least 55° C., at least 60° C., at least 65° C. or less than 70° C. In an embodiment, the protease treatment, the proteolytic treatment may be suitably carried out for at least 4 hours, at least 5 hours, at least 6 hours, at least 7 hours, at least 8 hours, at least 9 hours, at least 10 hours, at least 11 hours or at least 12 hours. The proteolytic treatment may be suitably carried out for less than 48 hours, particularly less than 36 hours, more particularly less than 24 hours, and even more particularly less than 18 hours. After the proteolytic treatment, a heat treatment could be performed to inactivate all enzyme activity. The second product (i.e. the yeast cell wall derivatives) is then separated by centrifugation to produce a soluble extract enriched with mannan (α-mannan), and an insoluble cell wall product enriched in β-glucan. The cell wall product enriched in β-glucan is discarded while the soluble extract enriched with mannan fraction (i.e. the yeast-derived soluble mannan-oligosaccharides or the soluble yeast cell wall derivatives) is retained. These yeast-derived soluble mannan-oligosaccharides can then be dried, e.g., spray dried. The soluble yeast cell wall derivatives of the present disclosure can be further submitted to specific complementary treatments such as concentration or filtration.

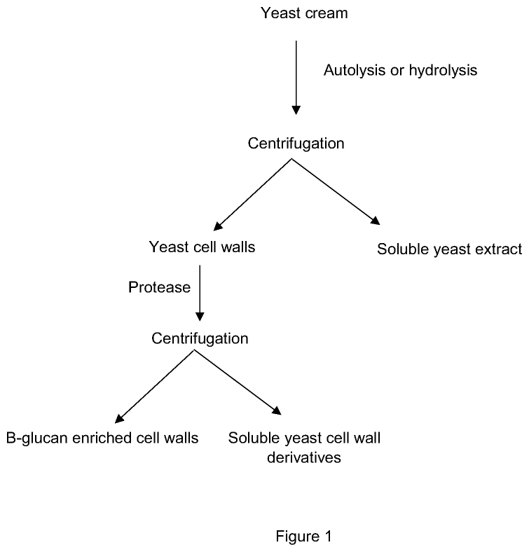

This exemplified process described above is shown in the flowchart of FIG. 1. Live yeast or cream yeast is subjected to autolysis and/or enzyme assisted hydrolysis in a process in which endogenous yeast enzymes and/or exogenous enzymes break down and solubilize some yeast macromolecules. In an embodiment, exogenous enzymes are added during the autolysis process. Soluble yeast extract is separated from insoluble yeast cell walls by centrifugation. The yeast cell walls are then treated with exogenous enzymes to further remove protein from the cell walls and thus degrade mannoproteins into mannopeptides which become release from the cell wall fractions. The β-glucan enriched cell walls are then separated and discarded from the secondary extract comprising the mannans by centrifugation, filtration or any other methods known in the art. Mannans, which have a high molecular weight, can be further purified and concentrated by ultrafiltration or any purification processes known in the art.

The soluble yeast cell wall derivatives of the present disclosure may therefore comprise soluble mannoproteins and/or soluble mannopeptides. The soluble mannopeptides may comprise O-linked mannan-oligosaccharides, i.e. are soluble O-linked mannopeptides.

The preparations of the present disclosure may be dried by any suitable process including, but not limited to, freeze-drying, roller drum drying, oven-drying, spray-drying, ring-drying, and combinations thereof and/or dried using film-forming equipment, and either may be used without further processing, or may be milled using any suitable technique.

Suitably, the hydrolysis of the yeast cell walls is carried out by adding hydrolases acting on peptide bonds. Such hydrolases are also called peptidases or proteases or proteolytic enzymes have number EC 3.4 in the EC classification. Peptidases catalyze the hydrolytic cleavage of the peptide bond (C—N). For example, the hydrolases of the present disclosure are chosen among exopeptidases, especially aminopeptidase, dipeptidase, dipeptidyl-peptidase, tripeptidyl-peptidase, peptidyl-dipeptitase, carboxypeptidase of serine type, carboxypeptidase of cysteine type, metallocarboxypeptidase, omega-peptidase, and endopeptidases (or proteinase). In particular, the hydrolase of the present disclosure is an endopeptidase (or proteinase). In an embodiment, the soluble yeast cell wall derivatives of the present disclosure are obtained by enzymatic hydrolysis of the yeast cell walls with at least one peptidase as, for example, papain, trypsin, chymotrypsin, subtilisin, pepsin, thermolysin, pronase, flavastacine, enterokinase, factor Xa protease, furin, bromelain, proteinase K, genenase I, thermitase, carboxypeptidase A, carboxypeptidase B, collagenase, Alcalase®, Neutrase®, or combinations thereof. The conditions of use of the enzymes (in particular, their concentration, duration of hydrolysis, temperature) can be easily determined by the person skilled in the art.

The soluble yeast cell wall derivatives of the present disclosure may consist of, or comprise, a yeast-derived soluble mannan oligosaccharide product. The yeast-derived soluble mannan-oligosaccharide product in accordance with the present disclosure may also be characterized as comprising at least 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan or mannan-oligosaccharide, preferably at least 30% mannan or mannan-oligosaccharide, and more preferably at least 50% mannan or mannan-oligosaccharide. The yeast-derived soluble mannan-oligosaccharide product in accordance with the present disclosure may be characterized as comprising at least 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% protein. The total mannan or mannan oligo-saccharide and protein in the yeast-derived soluble mannan-oligosaccharide product may not exceed 100%.

The soluble yeast cell wall derivatives of the present disclosure may consist of, or comprise, a yeast-derived soluble mannan oligosaccharide product. The yeast-derived soluble mannan-oligosaccharide product in accordance with the present disclosure may also be characterized as comprising a ratio of soluble mannan-oligosaccharides/proteins of 70/30, 69/31, 68/32, 67/33, 66/34, 65/35, 64/36, 63/37, 62/38, 61/39, 60/40, 59/41, 58/42, 57/43, 56/44, 55/45, 54/46, 53/47, 52/48, 51/49, 50/50, 49/51, 48/52, 47/53, 46/54, 45/55, 44/56, 43/57, 42/58, 41/59, 40/60, 39/61, 38/62, 37/63, 36/64, 35/65, 34/66, 33/67, 32/68, 31/69, 30/70, 29/71, 28/72, 27/73, 26/72, 25/75, 24/76, 23/77, 22/78, 21/79, 20/80, 19/81, 18/82, 17/83, 16/84 or 15/85.

In an embodiment, the yeast-derived soluble mannan-oligosaccharide product may be also characterized as comprising at least 20%, and preferably at least 30% mannan or mannan-oligosaccharide, at least 35% mannan-oligosaccharide, at least 40% mannan-oligosaccharide, at least 45% mannan-oligosaccharide, at least 50% mannan-oligosaccharide, at least 60% mannan-oligosaccharide or at 65% least mannan-oligosaccharide. In another embodiment, the yeast-derived soluble mannan-oligosaccharide product may be also characterized as comprising at least 70%, 65%, 60%, 55%, 50%, 45%, 40%, 35%, 30%, and preferably at least 35% protein. In particular, the yeast-derived soluble mannan-oligosaccharide of the present disclosure does not comprise or contain soluble yeast extract.

The soluble yeast cell wall derivatives of the present disclosure may be characterised as comprising less than 70% mannan-polysaccharide, such as less than 65%, 60%, 55%, 50%, 45%, 40%, 35%, 30%, 25%, 20%, 15%, 10%, 9%, 8%, 7%, 6%, 5%, 4%, 3%, 2% or 1% mannan-polysaccharide.

The percentages (%) provided herein are typically by mass on a dry matter basis. The percentages (%) provided herein may be a % of the soluble yeast cell wall derivative as a whole. The sum of the components within the yeast-derived soluble mannan-oligosaccharide product may not exceed 100%.

The soluble yeast cell wall derivatives of the present disclosure may be obtainable by hydrolysing yeast cell walls. The yeast cell walls may be insoluble yeast cell walls. The hydrolysis may be carried out via enzymatic treatment of yeast cell walls. The enzymatic treatment may be protease treatment. Suitable proteases are discussed in more detail above. The soluble yeast cell wall derivatives of the present disclosure may be obtainable by hydrolysing yeast cell walls and retaining the soluble fraction, e.g. the soluble mannan-oligosaccharide fraction or the soluble mannopeptide fraction.

The preparation in accordance with the present disclosure is for biologically treating organic or plant matter such as, for example leaf litter or crop residues, to accelerate its degradation or decomposition and, consequently, reduce the overwintering of the inoculum.

In one aspect, the present method uses soluble yeast cell wall derivatives as described herein to modify the structure of organic matter or plant matter so that the degradable structural components comprised in the organic or plant matter is essentially turned into a decomposition product.

By “organic or bioorganic matter”, it is included any organic (i.e. carbon-containing) matter derived from or produced by a biological organism such as a plant.

As used herein, the term “plant matter” includes any matter (such as foliage, leaves, straw, crop residues, leaf litter, fruits, flowers, grain and seeds) derived from or produced by plants. A range of plant matter may be used in the method of the present disclosure such as plant matter from monocotyledonous-derived materials (straw and bran from wheat, barley, rice, oats, rye, sugar cane, corn, corn stover, corn stalks, Brewer's grain, grass, hay, field waste and the like); non-graminaceous monocotyledonous-based waste (for example, from asparagus); dicotyledonous-derived materials (for example fruit and vegetable wastes; crop residues from vegetables and legumes; fruit and stone fruit trees crop residues (such as leaves, fallen leaves, leaf litter, fruits or the like) from apple trees, pear trees, vines, grapes, cherry trees, plum trees, apricot trees, peach trees, citrus trees and the like. The organic or plant matter may comprise monocotyledonous plant matter or dicotyledonous plant matter. The monocotyledonous plant matter or dicotyledonous plant matter may comprise leaves, tree foliage, leaf litter and/or crop residues of, for example, cereals crops, sugar cane, corn, vines, vegetable crops or fruit trees. The organic or plant matter may comprise leaves and/or leaf litter from apple trees. The organic or plant matter may comprise crop stubble. The soluble yeast cell wall derivatives of the present disclosure may be contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble yeast cell wall derivatives are contacted with tree foliage or leaves at about between 30% and 75% leaf fall; and/or the soluble yeast cell wall derivatives are contacted with plant matter on the ground at about 30%, 35%, 40%, 45%, 50%, 55%, 60%, 65%, 70%, 75%, 80%, 85%, 90% or 95% leaf fall.

The soluble yeast cell wall derivatives of the present disclosure are capable of degrading or decomposing or accelerating the degradation or the decomposition (i.e. breaking down) of one or more components of the organic matter or plant matter (such as, for example, leaf litter or crop residue), thereby altering the chemical composition and/or physical structure of the organic matter or plant matter. For example, the soluble yeast cell wall derivatives of the present disclosure capable of degrading components of organic matter or plant matter may be used to alter and/or reduce the overall level of structure in the matter resulting into decomposition product. The rate of degradation or decomposition may vary depending on number of factors including, but not limited to, the composition of the organic or plant matter and the conditions in which the soluble yeast cell wall derivatives are contacted with the organic or plant matter (time or period of application, temperature or hydration (water-content) levels).

Without wishing to be bound by theory, it is believed that the soluble yeast cell wall derivatives of the invention stimulate a resident microbial population on the organic matter or plant matter (such as leaf litter or crop residue), and organic matter or plant matter degrading fungi in the soil (such as leaf litter degrading fungi). The resident microbial populations and leaf degrading fungi are capable of degrading or decomposing, or accelerating the degradation or the decomposition of one or more components of the organic matter or plant matter.

By “decomposition product”, it is included matter derived from the organic or plant matter which contains one or more components of the organic or plant matter that has not been degraded or fully degraded by the soluble yeast cell wall derivatives. In other words, “decomposition product” includes partially decomposed organic or plant matter.

In one embodiment, the preparation in accordance of the present disclosure is for biologically treating leaf litter of apple tree to accelerate its degradation or decomposition and, consequently, reduce the Venturia inaequalis overwintering of the inoculum (e.g. reduce the number of ascospores and/or pseudothecia present and pseudothecium fertility). Accordingly, the preparation in accordance with the present disclosure demonstrates a comparable performance to urea used in conventional orchards for improved sanitation (i.e. for management of overwintering inoculum of the apple scab pathogen, V. inaequalis, and hasten leaf degradation).

The soluble yeast cell wall derivatives of the present disclosure may be for reducing the inoculum of an overwintering pathogenic fungus in organic matter or plant matter. The soluble yeast cell wall derivatives of the present disclosure may be for reducing the number of ascospores and/or pseudothecia in organic matter or plant matter. The soluble yeast cell wall derivatives of the present disclosure may be for reducing the incidence of apple scab in organic matter or plant matter, e.g. the incidence of apple scab in an orchard. The soluble yeast cell wall derivatives of the present disclosure may be for managing pathogenic fungi.

The soluble yeast cell wall derivatives of the present disclosure increase the rate at which organic and plant matter are degraded. In one aspect, this results in a lower availability of nutrients for pathogenic fungi, such as overwintering pathogenic fungi, such that the total inoculum of the fungi in organic or plant matter treated with the soluble yeast cell wall derivatives is reduced when compared to untreated organic or plant matter. The soluble yeast cell wall derivatives of the present disclosure may therefore be used for managing overwintering pathogenic fungi generally. The overwintering pathogenic fungi is preferably apple scab, V. inaequalis, but may be any pathogenic fungi which can overwinter on dead plant materials of perennial crops such as powdery mildew, brown rot and grey mold. Powdery mildew fungi include Erysiphe necator (grapevines), brown rot fungi include Monilinia taxa; Monilinia fructicola; Monilinia fructigena and grey mold fungi include Botrytis cinerea (an anamorph of Botryotinia fuckiliana.

The preparation in accordance with the present disclosure is applied to the plant matter (leaves, tree foliage, leaf litter and/or crop residues) in an amount of at least about 0.5, 1, 2, 3, 4, 5, 6, 7, 8, 9, 10, 11, 12, 13, 14, 15, 16, 17, 18, 18, 20, 21, 22, 23, 24, 25 or more than 26 kg of dry matter by hectare. In an embodiment, the preparation in accordance with the present disclosure is applied to the plant matter (leaves, tree foliage, leaf litter and/or crop residues) in an amount of at least 1 to 20; 1 to 15; 1 to 10; 1 to 5 or 5 to 10 kg of dry matter by hectare. In another embodiment, the preparation in accordance with the present disclosure is applied to the plant matter (leaves, tree foliage, leaf litter and/or crop residues) in an amount of 1 to kg of dry matter by hectare.

In an embodiment, the preparation in accordance of the present disclosure can be applied to the tree foliage or leaves (before leaf fall) at about 10%, 15%, 20%, 25%, 30%, 35%, 40%, 45%, 50%, 55%, 60%, 65%, 70%, 75%, 80%, 85%, 90% or 95% leaf fall. In an embodiment, the preparation in accordance of the present disclosure can be applied to the tree foliage or leaves (before leaf fall) at about between 30% and 90% leaf fall. Alternatively, the preparation in accordance of the present disclosure can be applied to the tree foliage or leaves at about between 30% and 75% leaf fall. In another embodiment, the preparation in accordance of the present disclosure is applied to the plant matter on the ground at about 30%, 35%, 40%, 45%, 50%, 55%, 60%, 65%, 70%, 75%, 80%, 85%, 90% or 95% leaf fall. For example, in orchards, the preparation of the present disclosure can be applied after fruit harvest but before leaf fall (e.g. at about 10%, 15%, 20%, 25%, 30%, 35%, 40%, 45%, 50%, 55%, 60%, 65%, 70%, 75%, 80%, 85%, 90% or 95% leaf fall) and/or on the plant matter on the ground at about 30%, 35%, 40%, 45%, 50%, 55%, 60%, 65%, 70%, 75%, 80%, 85%, 90% or 95% leaf fall.

The preparation according to the present disclosure can be used alone, in combination, i.e. simultaneously, or in alternation, i.e. sequentially with another active ingredient such as urea.

In some embodiments, the preparation in accordance with the present disclosure may comprise an agriculturally acceptable carrier or a nutrient.

The word “comprising” in the claims may be replaced by “consisting essentially of” or with “consisting of”,” according to standard practice in patent law.

The following example serves to further describe and define the invention and is not intended to limit the invention in any way.

### EXAMPLES

### Example 1: Efficacy of Soluble Yeast Cell Wall Derivatives on Apple Leaf Degradation

The objective of this trial was to establish the potential of soluble yeast cell wall derivatives to accelerate degradation of apple tree leaves on the orchard floor thereby reducing overwintering potential of apple scab (Venturia inaequalis).

Leaf Populations:

At 95% leaf drop, two populations of autumn leaves were collected: 1) yellow leaves (yellow leaves ready to fall; harvested from the treatment trees and placed into wire grids; and 2) brown leaves (brown leaves which have fallen but not yet showing visible degradation collected from the orchard floor and placed into wire grids).

Treatments:

The following treatments were tested: 1) untreated control (no application); 2) water control (1000 L water per hectare); 3) urea (50 g per 1000 L per hectare); and 4) soluble yeast cell wall derivatives (Lallemand) (5 kg dry matter/ha; 10 L/ha in 1000 L water per hectare).

Treatments were applied to the soil using a non-mechanised back pack sprayer as applicator, in a one meter radius around the tree stem (grids were placed within this circumference).

Treatment application times: 1) 95% leaf drop; 2) 95% leaf drop+two weeks; and 3) 95% leaf drop+four weeks.

Examination Parameters:

All evaluations were done separately on each of the two leaf populations selected at the start of the trial.

Visual In-Field Leaf Degradation Rating Per Grid:

Leaf degradation according to a 0-9 scale, on a per grid estimate, reflecting the surface area which has decomposed; with “0” representing no degradation, “3” being approx. 30% degradation, “6” representing 60% degradation, and “9” complete degradation.

Visual Leaf Degradation Rating Per Leaf:

Leaf degradation rating of each leaf, according to a 0-9 scale, reflecting the surface area which has decomposed; with “0” representing no degradation, “3” being approx. 30% degradation, “6” representing 60% degradation, and “9” complete degradation.

Leaf Area Degradation:

Leaf degradation was expressed as the reduction in leaf area (mm²) based on readings taken with a leaf area scanner (LI-COR, LI-3050C Transparent Belt Conveyor). Samples were placed onto a transparent conveyor belt which allowed the leaf to pass through the scanning head. The accumulated leaf area was determined by passing all leaves of a grid through the scanner and subsequently an average per leaf area (mm 2).

Results:

TABLE 1
Average leaf degradation score in the sampling grids (on a
scale of 0 to 9) per treatment, recorded every two weeks over
an eight-week period for the population of yellow leaves

| | Degradation category (scale 0-9) per assessment stage | | | | |
|---|---|---|---|---|---|
| | (weeks after the last application) | | | | |
| Treatment | 4 weeks | 6 weeks | 8 weeks | 10 weeks | 12 weeks |
| 1) untreated | 1.2a¹ | 1.7 a | 2.8 a | 3.2 a | 5.0ab |
| control | (+20%)² | (−6%) | (0%) | (−9%) | (+6%) |
| 2) water | 1.0 a | 1.8 a | 2.8 a | 3.5 a | 4.7 a |
| control | (0%) | (0%) | (0%) | (0%) | (0%) |
| 3) urea | 1.3a | 2.5a | 3.2a | 3.8 a | 5.3ab |
| | (+30%) | (+39%) | (+14%) | (+9%) | (+13%) |
| 4) soluble | 2.8b | 4.2b | 5.0b | 5.5b | 6.8b |
| yeast cell | (+180%) | (+133%) | (+100%) | (+57%) | (+45%) |
| wall | | | | | |
| derivatives | | | | | |

¹Values in the same column followed by different letters indicate significant differences (P < 0.05, P < 0.01, P < 0.001) according to the Tukey HSD test. One-way ANOVA table with a completely randomized block design. Values in red indicate where significant differences were expressed.
²Values in parentheses indicate the percentage by which leaf degradation of a treatment exceeded the water control

As shown in Table 1, leaf degradation was significantly enhanced on yellow leaves treated with soluble yeast cell wall derivatives, compared to the water control, as well as the application of urea, across all assessment stages. Leaves within the sample grid sprayed with water, or left untreated, exhibited the lowest degradation rating across all assessment stages. In comparison to the water control, treatment of the leaves within a sample grid with soluble yeast cell wall derivatives, improved degradation by 180% to 45%, respectively for the 4 week and 12 week assessments after the last application. In comparison, leaf degradation for urea application exceeded the water control by 30% and 9%, for the 4 week and 12 week assessments, respectively.

TABLE 2
Average leaf degradation score in the sampling grids (on a
scale of 0 to 9) per treatment, recorded every two weeks over
an eight-week period for the population of brown leaves

| | Degradation category (scale 0-9) per assessment stage | | | | |
|---|---|---|---|---|---|
| | (weeks after the last application) | | | | |
| Treatment | 4 weeks | 6 weeks | 8 weeks | 10 weeks | 12 weeks |
| 1) untreated | 1.0 | 1.2 a¹ | 1.8 a | 2.8 a | 3.8 a |
| control | (+43%)² | (0%) | (−10%) | (−13%) | (−12%) |
| 2) water | 0.7 | 1.2 a | 2.0 a | 3.2 a | 4.3 a |
| control | (0%) | (0%) | (0%) | (0%) | (0%) |
| 3) urea | 1.0 | 2.2 ab | 3.2 ab | 4.3 ab | 5.7 ab |
| | (+43%) | (+83%) | (+60%) | (+34%) | (+33%) |
| 4) soluble | 1.5 | 3.5 b | 4.2 b | 5.7 b | 7.2 b |
| yeast cell | (+114%) | (+192%) | (+110%) | (+78%) | (+67%) |
| wall | | | | | |
| derivatives | | | | | |

¹Values in the same column followed by different letters indicate significant differences (P < 0.05, P < 0.01, P < 0.001) according to the Tukey HSD test. One-way ANOVA table with a completely randomized block design. Values in red indicate where significant differences were expressed.
²Values in parentheses indicate the percentage by which leaf degradation of a treatment exceeded the water control

As shown in Table 2, treatment effects were essentially similar for degradation of brown leaves than degradation of yellow leaves. More particularly, leaf degradation was significantly enhanced on brown leaves treated with soluble yeast cell wall derivatives, compared to the water control, as well as the application of urea. Leaves within the grid sprayed with water, or left untreated, exhibited the lowest degradation rating across all assessment stages. In comparison to the water control, treatment of the leaves within a sample grid with yeast-derived soluble mannan-oligosaccharides, improved degradation by 192% to 67%, respectively for 6 week and 12 week assessments after the last treatment. In comparison, leaf degradation for urea application exceeded the water control by 83% to 33%, for the 6 week and 12 week assessments, respectively.

TABLE 3
Reduction in leaf area (mm²) at all assessment stages, by treatment,
recorded after collection of orchard leaf grids, measured by the
leaf area scanning method, for yellow leaves and brown leaves

Average leaf area reduction (mm²)

| Treatment | yellow leaves | brown leaves |
|---|---|---|
| water control | 13.7 ab¹ | 9.3 ab |
| | (0%) | (0%) |
| urea | 15.0 abc | 14.1 bc |
| | (+10%) | (+52%) |
| soluble yeast cell wall | 17.4 c | 14.5 c |
| derivatives | (+27%) | (+56%) |

¹Values in the same column followed by different letters indicate significant differences (P < 0.05, P < 0.01, P < 0.001) according to the Tukey HSD test. One-way ANOVA table with a completely randomized block design. Values in red indicate where significant differences were expressed

As shown in Table 3, leaf area was significantly reduced by application of soluble yeast cell wall derivatives, compared to the water control, for both yellow and brown leaves. Albeit not significant, application of urea also reduced the leaf area compared to the water control. Yellow leaves treated with soluble yeast cell wall derivatives outperformed the water control by 27%, while by 56% on brown leaves.

In conclusion, for this first trial, it was observed that leaf degradation of up to 30%, 50% and 80%, required 12 weeks after the last application of water, urea and soluble yeast cell wall derivatives, respectively due to cold weather conditions and lack of rains. However, in these conditions, the leaf degradation by soluble yeast cell wall derivatives was significantly better than the untreated control as well as urea.

A second and third trials were conducted to further study the potential of soluble yeast cell wall derivatives to accelerate decomposition of apple leaves on orchard floor and to determine the effect of leaf decomposition on V. inaequalis ascospores presence and release from leaves on the orchard floor, respectively.

Leaf Populations:

At 95% leaf drop, yellow stage, scab infected apple leaves were placed on the cleared orchard floor and kept in place by shade net fastened to the orchard floor using nails. This was to ensure sufficient contact between leaves and soil.

Treatments:

The following treatments were tested: 1) untreated control; and 2) soluble yeast cell wall derivatives.

For the second trial (i.e. leaf degradation), the treatments were applied to a 1 square meter area on and around leaf grids, using a hand-held high pressure sprayer. For each treatment there were 8 replicates.

For the third trial (i.e. effect of leaf degradation on ascospore presence and release from leaves), the treatments were applied using a mechanised backpack sprayer covering the entire orchard floor in rows were leaf grids where placed, and later the seedling trees. For the ascospore release assessments, there were 5 replicates.

Treatment application times: 1) 95% leaf drop; 2) 95% leaf drop+two weeks; and 3) 95% leaf drop+four weeks.

Examination Parameters:

Leaf degradation evaluations were done on a fortnightly basis (every two weeks) until leaf degradation ratings of 6 or above, on the 0-9 scale, were reached by at least one treatment.

Ascospore incidence was done twice during early spring period (i.e. after the overwintering period) according to the waterbath method.

Visual Leaf Degradation Rating:

Leaf degradation according to a 0-9 scale reflecting the surface area which has decomposed; with “0” representing no degradation, “3” being about 30% degradation, “6” being 60% degradation, and “9” complete degradation.

At the end of the trial, leaves were collected for a more detailed rating of each leaf in the grid. Leaves were grouped into each category from 0 to 9, expressed as percentage of total leaves falling into relevant category.

To condense these results into a single value, a degradation index was calculated based on the number of leaves in each category, using the following calculation:

Degradation index=(C0×0)+(C1×1)+(C2×2)+(C3×3)+(C4×4)+(C5×5)+(C6×6)+(C7×7)+(C8×8)+(C9×9)/9

(with C0=percentage of leaves at Class 0; C1=percentage of leaves at Class 1, etc.)

Ascospore Incidence:

Ascospore incidence was executed as per the waterbath method of A. Kollar, 2000 and CEHM, 2004 at start of trial (prior to treatment application). Three weeks later, a mid-assessment was done and three weeks later a final assessment was performed. At the first assessment two weeks after the last application, no ascospores were detected, however conidia counts were quantified.

Results:

TABLE 4
Average grid degradation ratings (on a scale of
0 to 9) per treatment, recorded every two weeks

Examination timing

| | 2 weeks after | 4 weeks after |
|---|---|---|
| Treatment | last application | last application |
| untreated control | 2.4a¹ | 3.1a |
| soluble yeast cell | 4.1b | 4.9a |
| wall derivatives | | |

¹Values in the same row followed by different letters indicate significant differences (P < 0.05) according to the Tukey HSD test. One-way ANOVA table with a completely randomized block design

As shown in Table 4, leaf degradation was markedly quicker than the first trial due, in part, to warmer conditions and rain. At the first examination (i.e. two weeks after final application), leaves treated with soluble yeast cell wall derivatives were significantly more degraded than untreated leaves.

Initial assessments according to the waterbath method, prior to treatment application, showed that 5925 conidia/millilitre were identified on untreated leaves at start of trial. Two weeks after the final treatment application, treated leaves showed a significant reduction in conidia counts, most likely as a result of leaf degradation, with leaves treated with soluble yeast cell wall derivatives showing the lowest number of conidia (200 conidia/ml), followed by the untreated leaves (1125 conidia/ml).

In conclusion, results indicated the soluble yeast cell wall derivatives are a good alternative to accelerate leaf degradation on the orchard floor, especially for organic production or where urea usage is to be minimized.

### Example 2: Efficacy of Soluble Yeast Cell Wall Derivatives on Ascospore Projection

The objective of this trial was to evaluate the efficacy of the soluble yeast cell wall derivatives on their ability to reduce the apple scab inoculum (i.e. ascospores) on overwintering leaves.

The trial was carried out on a “Gala” apple variety in an orchard severely affected by apple scab.

The following treatments were tested: M1: untreated control; M2: urea, one application at (30% fallen leaves); M3: soluble yeast cell wall derivatives (Lallemand), one application at 5 l/ha (30% fallen leaves); M4: soluble yeast cell wall derivatives, one application at 10 l/ha (30% fallen leaves); M5: soluble yeast cell wall derivatives, two applications at 10 l/ha (first application at beginning of leaf drop and second application at 30% fallen leaves). Each treatment had three replicates.

One batch of 100 g of leaves per replication was placed in plastic mesh bags with fine mesh on the outdoor ground, in orchard conditions. Treatments were applied in November to the trees using a backpack sprayer (SOLO) at a volume of 1000 L/ha. The leaves harvested for the tiral were heavily contaminated with apple scab, since they had served as an untreated control in an in-season scab test.

The ascospore projection of Venturia inaequalis was evaluated in March. Ascospores were count on a Malassez cell after an artificial projection was made following the Kollar waterbath protocol (1998). The results were presented as the number of ascospores/μl.

All meteorological records came from the SudAgroMétéo station on the SudExpé Marsillargues site. These data were recorded throughout the duration of the test. The results were analyzed using a one-way ANOVA and multiple comparisons of the mean were analysed using a Tukey post-hoc test at 5% threshold.

Results:

An apple scab ascospore count was performed using the modified Kollar protocol in March. This count was used to determine the average concentration of ascospores present in the leaf litter according to the modality.

TABLE 5
Average concentration of apple scab ascospores
discharged in the overwintered leaves

| | Average concentration | Homogeneous |
|---|---|---|
| Treatment | (ascospores/μl) | group |
| M1: untreated control | 139 | a¹ |
| M2: urea, one application at | 39 | b |
| 50 kg/ha | | |
| M3: soluble yeast cell wall | 49 | b |
| derivatives, one application | | |
| at 5 l/ha | | |
| M4: soluble yeast cell wall | 53 | b |
| derivatives, one application | | |
| at 10 l/ha | | |
| M5: soluble yeast cell wall | 46 | b |
| derivatives, two applications | | |
| at 10 l/ha | | |

| | |
|---|---|
| P-value | 0.00130993 |
| Tukey Test | Highly significant |

¹The letters indicate the statistically homogeneous groups (Tukey test, alpha threshold = 5%).

As shown in Table 5, the concentrations of apple scab ascospores of the modalities treated with soluble yeast cell wall derivatives were not significantly different from the modality with urea independently of the dose of soluble yeast cell wall derivatives and the number of applications, and significantly lower than that of the untreated control. The use of soluble yeast cell wall derivatives has significantly reduced the primary inoculum, which will reduce the disease pressure in the orchard.

### Example 3: Preparation of the Soluble Yeast Cell Wall Derivatives According to the Present Invention

This example describes typical preparation of soluble yeast cell wall derivatives as described in Examples 1 and 2.

Industrial cream yeast (20% of dry matter) comprising whole yeast cells from a yeast strain of S. cerevisiae was subjected to a heat treatment at a temperature of at least 40° C. for 3 to 20 hours. After this treatment of autolysis/hydrolysis, the resulting hydrolysate was subjected to several steps of centrifugation to separate the soluble yeast extract fraction from the insoluble yeast cell wall fraction. The insoluble yeast cell wall fraction was recovered and hydrolysed by the addition of at least one exogenous protease and incubated during at least 2 hours at a temperature of at least 40° C. For example, the protease was used at a concentration of 0.01% to 1% (weight/weight). The cell wall fraction was then separated from the solubilized fraction hydrolysed from the insoluble fraction (ie. the soluble yeast cell wall derivatives) by several steps of centrifugation and washing with water. The solubilised hydrolysed fraction was purified and concentrated by ultrafiltration and dried.

### REFERENCES

Burchill, R. T., Hutton, K. E., Crosse, J. E. & Garrett, C. M. E. (1965). Inhibition of the perfect stage of Venturia inaequalis (Cooke) Wint. by urea. Nature 205: 520-521.
Kollar, A., 1998. A simple method to forecast the ascospore discharge of Venturia inaequalis/Eine einfache Methode zur Vorhersage der Ascosporenausschleuderung von Venfuria inaequalis. Zeitschrift für Pflanzenkrankheiten and Pflanzenschutz/Journal of Plant Diseases and Protection, pp. 489-495. Porsche, F. M., A.-C. Hahn, B. Pfeiffer and A. Kollar. 2016. Yeast extract applications to reduce the primary inoculum of Ventirua inaequalis. In Eco-Fruit: Proceedings of the 17th International Conference on Organic Fruit Growing from Feb. 15 to Feb. 17, 2016 at the University of Hohenheim, Germany (ed. by Fördergemeinschaft Ökologischer Obstabau e. V., Weinsberg, pp. 53-59.
Porsche, F. M., B. Pfeiffer and A. Kollar. 2017. A new phytosanitary method to reduce the ascospore potential of Ventirua inaequalis. Plant Disease 101: 414-420.

While the invention has been described in connection with specific embodiments thereof, it will be understood that the scope of the claims should not be limited by the preferred embodiments set forth in the examples, but should be given the broadest interpretation consistent with the description as a whole.

Further Aspects of the Invention:

1. A method for accelerating decomposition of organic or plant matter comprising contacting the organic or the plant matter with an effective amount of soluble yeast cell wall derivatives as an active ingredient for degrading the organic or plant matter to produce a decomposition product.

2. The method of aspect 1, wherein said soluble yeast cell wall derivatives comprise a yeast-derived soluble mannan-oligosaccharide fraction.

3. The method of aspect 2, wherein the yeast-derived soluble mannan-oligosaccharide fraction comprises
(a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan, preferably wherein the yeast-derived soluble mannan-oligosaccharide product comprises at least about 20% or at least about 30% mannan; and
(b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably wherein the yeast-derived soluble mannan-oligosaccharide product comprises at least about 30% or 35% of proteins.

4. The method of any one of aspects 1 to 3, wherein said organic or plant matter comprises monocotyledonous plant matter or dicotyledonous plant matter, preferably wherein said monocotyledonous plant matter or dicotyledonous plant matter comprises leaves, tree foliage, leaf litter and/or crop residues.

5. The method of aspect 4, wherein said organic or plant matter comprises leaves, leaf litter and/or crop residues of cereals crops, sugar cane, corn, vines, vegetable crops or fruit trees.

6. The method of aspect 5, wherein said organic or plant matter comprises leaves and/or leaf litter from apple trees.

7. The method of any one of aspects 4 to 6, wherein
(a) said soluble yeast cell wall derivatives are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble yeast cell wall derivatives are contacted with tree foliage or leaves at about between 30% and 75% leaf fall; and/or
(b) said soluble yeast cell wall derivatives are contacted with plant matter on the ground at about 30%, 35%, 40%, 45%, 50%, 55%, 60%, 65%, 70%, 75%, 80%, 85%, 90% or 95% leaf fall.

8. The method of any one of aspects 1 to 7, wherein the soluble yeast cell wall derivatives are used in alone or in combination with urea.

9. Use of soluble yeast cell wall derivatives for degrading organic or plant matter to produce a decomposition product.

10. The use of aspect 9, wherein said soluble yeast cell wall derivatives are a yeast-derived soluble mannan-oligosaccharide product which comprises
(a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan, preferably at least about 20% or at least about 30% mannan; and
(b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins, preferably at least about 30% or 35% of proteins.

11. The use of aspect 9 or 10, wherein said organic or plant matter comprises monocotyledonous plant matter or dicotyledonous plant matter, preferably wherein said monocotyledonous plant matter or dicotyledonous plant matter comprises leaves, tree foliage, leaf litter and/or crop residues.

12. The use of aspect 11, wherein said organic or plant matter comprises leaves, leaf litter and/or crop residues of cereals crops, sugar cane, corn, vines, vegetable crops or fruit trees.

13. The use of aspect 12, wherein said organic or plant matter comprises leaves and/or leaf litter from apple trees.

14. The use of any one of aspects 11 to 13, wherein
(a) said soluble yeast cell wall derivatives are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble yeast cell wall derivatives are contacted with tree foliage or leaves at about between 30% and 75% leaf fall; and/or
(b) said soluble yeast cell wall derivatives are contacted with plant matter on the ground at about 30%, 35%, 40%, 45%, 50%, 55%, 60%, 65%, 70%, 75%, 80%, 85%, 90% or 95% leaf fall.

15. The method of any one of aspects 1 to 8 or the use of any one of aspects 9 to 14, wherein said soluble yeast cell wall derivatives are obtained by a method comprising the following steps:
i. providing a yeast cell material from a species from the genera Saccharomyces, Kluyveromyces, Hanseniaspora, Metschnikowia, Pichia, Starmerella, Torulaspora or Candida, preferably wherein the yeast is S. cerevisiae;
ii. subjecting said yeast material to autolysis and/or enzyme assisted hydrolysis for a sufficient time to obtain a yeast autolysate and/or a yeast hydrolysate comprising a soluble yeast extract fraction and an insoluble yeast cell wall fraction;
iii. subjecting said yeast autolysate or said yeast hydrolysate to separation to separate the soluble yeast extract fraction from the insoluble yeast cell wall fraction;
iv. recovering the yeast cell wall fraction and discarding the soluble yeast extract fraction;
v. subjecting the yeast cell wall fraction to an enzymatic treatment with a protease to obtain yeast cell wall derivatives comprising a β-glucan enriched cell wall fraction and a yeast-derived soluble mannan-oligosaccharide fraction;
vi. separating said β-glucan enriched cell wall fraction from said yeast-derived soluble mannan-oligosaccharide fraction; and
vii. recovering said yeast-derived soluble mannan-oligosaccharide fraction.

1. A method for accelerating decomposition of organic or plant matter comprising contacting the organic or the plant matter with an effective amount of soluble hydrolysed yeast cell wall derivatives as an active ingredient for degrading the organic or plant matter to produce a decomposition product.

2. The method of claim 1, wherein the soluble hydrolysed yeast cell wall derivatives are soluble enzymatically-treated yeast cell wall derivatives.

3. The method of claim 2, wherein the soluble enzymatically-treated yeast cell wall derivatives are soluble protease-treated yeast cell wall derivatives.

4. The method of claim 1, wherein said soluble hydrolysed yeast cell wall derivatives comprise or consist of a soluble mannan-oligosaccharide fraction.

5. The method of claim 4, wherein the yeast-derived soluble mannan-oligosaccharide fraction comprises:
(a) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide by mass on a dry matter basis, preferably wherein the soluble mannan-oligosaccharide fraction comprises at least about 20% or at least about 30% mannan-oligosaccharide; and
(b) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins by mass on a dry matter basis, preferably wherein the soluble mannan-oligosaccharide fraction comprises at least about 30% or 35% of proteins.

6. The method of claim 1, wherein said organic or plant matter comprises monocotyledonous plant matter or dicotyledonous plant matter, preferably wherein said monocotyledonous plant matter or dicotyledonous plant matter comprises leaves, tree foliage, leaf litter and/or crop residues.

7. The method of claim 6, wherein said organic or plant matter comprises leaves, leaf litter and/or crop residues of cereals crops, sugar cane, corn, vines, vegetable crops or fruit trees.

8. The method of claim 7, wherein said organic or plant matter comprises leaves and/or leaf litter from apple trees.

9. The method of claim 6, wherein
(a) said soluble hydrolysed yeast cell wall derivatives are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble hydrolysed yeast cell wall derivatives are contacted with tree foliage or leaves at about between 30% and 75% leaf fall; and/or
(b) said soluble hydrolysed yeast cell wall derivatives are contacted with plant matter on the ground at about 30%, 35%, 40%, 45%, 50%, 55%, 60%, 65%, 70%, 75%, 80%, 85%, 90% or 95% leaf fall.

10. The method of claim 1, wherein the soluble hydrolysed yeast cell wall derivatives are used alone or in combination with urea.

11-16. (canceled)

17. The method of claim 1, wherein the soluble hydrolysed yeast cell derivatives are obtainable by hydrolysing a yeast cell wall fraction.

18. The method of claim 17, wherein the soluble hydrolysed yeast cell derivatives are obtainable by hydrolysing a yeast cell wall fraction with an enzyme, optionally with a protease.

19. The method of claim 1, wherein said soluble hydrolysed yeast cell derivatives are obtainable by:
i. subjecting a yeast cell wall fraction to an enzymatic treatment to obtain insoluble yeast cell wall derivatives comprising a β-glucan enriched cell wall fraction and a yeast-derived soluble mannan-oligosaccharide fraction; and
ii. separating said β-glucan enriched cell wall fraction from said yeast-derived soluble mannan-oligosaccharide fraction.

20. The method of claim 19, wherein the enzymatic treatment is protease treatment.

21. The method of claim 1, wherein said soluble hydrolysed yeast cell wall derivatives are obtainable by a method comprising the following steps:
i. providing a yeast cell material from a species from the genera Saccharomyces, Kluyveromyces, Hanseniaspora, Metschnikowia, Pichia, Starmerella, Torulaspora or Candida, preferably wherein the yeast is S. cerevisiae;
ii. subjecting said yeast material to autolysis and/or enzyme assisted hydrolysis for a sufficient time to obtain a yeast autolysate and/or a yeast hydrolysate comprising a soluble yeast extract fraction and an insoluble yeast cell wall fraction;
iii. subjecting said yeast autolysate or said yeast hydrolysate to separation to separate the soluble yeast extract fraction from the insoluble yeast cell wall fraction;
iv. recovering the yeast cell wall fraction and discarding the soluble yeast extract fraction;
v. subjecting the yeast cell wall fraction to an enzymatic treatment with a protease to obtain yeast cell wall derivatives comprising a β-glucan enriched cell wall fraction and a soluble mannan-oligosaccharide fraction;
vi. separating said β-glucan enriched cell wall fraction from said soluble mannan-oligosaccharide fraction; and
vii. recovering said soluble mannan-oligosaccharide fraction.

22. A method for reducing the inoculum of an overwintering pathogenic fungus, comprising contacting organic matter or plant matter with an effective amount of soluble hydrolysed yeast cell wall derivatives as an active ingredient for degrading the organic or plant matter to produce a decomposition product and to reduce the inoculum of the overwintering pathogenic fungus.

23. The method of claim 22, wherein the organic or plant matter comprises:
(a) monocotyledonous plant matter or dicotyledonous plant matter, preferably wherein said monocotyledonous plant matter or dicotyledonous plant matter comprises leaves, tree foliage, leaf litter and/or crop residues; and/or
(b) leaves, leaf litter and/or crop residues of cereals crops, sugar cane, corn, vines, vegetable crops or fruit trees; and/or
(c) leaves and/or leaf litter from apple trees or grape vines.

24-25. (canceled)

26. The method of claim 22, wherein the overwintering pathogenic fungus is apple scab (e.g. Ventirua inaequalis), powdery mildew (e.g. Erysiphe necator), brown rot (e.g. Monilinia laxa, Monilinia fructicola or Monilinia fructigena) or grey mold (e.g. Botrytis cinerea).

27. The method of claim 22, wherein:
(a) said soluble hydrolysed yeast cell wall derivatives are contacted with tree foliage or leaves at about 10%, 15%, 20%, 25%, 30%, 35%, 40%, 45%, 50%, 55%, 60%, 65%, 70%, 75%, 80%, 85%, 90% or 95% leaf fall, preferably wherein said soluble hydrolysed yeast cell wall derivatives are contacted with tree foliage or leaves at about between 30% and 75% leaf fall; and/or
(b) said soluble hydrolysed yeast cell wall derivatives are contacted with plant matter on the ground at about 30%, 35%, 40%, 45%, 50%, 55%, 60%, 65%, 70%, 75%, 80%, 85%, 90% or 95% leaf fall.

28. The method of claim 22, wherein the soluble hydrolysed yeast cell wall derivatives:
(a) are soluble enzymatically-treated yeast cell wall derivatives;
(b) are soluble protease-treated yeast cell wall derivatives;
(c) comprise or consist of a soluble mannan-oligosaccharide fraction; or
(d) comprise or consist of a soluble mannan-oligosaccharide fraction that comprises:
(i) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% mannan-oligosaccharide by mass on a dry matter basis, preferably wherein the soluble mannan-oligosaccharide fraction comprises at least about 20% or at least about 30% mannan-oligosaccharide; and
(ii) at least about 15%, 16%, 17%, 18%, 19%, 20%, 21%, 22%, 23%, 24%, 25%, 26%, 27%, 28%, 29%, 30%, 31%, 32%, 33%, 34%, 35%, 36%. 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or more than 60% of proteins by mass on a dry matter basis, preferably wherein the soluble mannan-oligosaccharide fraction comprises at least about 30% or 35% of proteins.

29-35. (canceled)
